(19) United States
(12) Patent Application Publication
(10) Pub. No.: US 2025/0172522 A1
(43) Pub. Date: May 29, 2025

(54) BIOSENSOR FOR TRIGLYCERIDES

(71) Applicant: UNIVERSITY OF THE WEST OF ENGLAND, BRISTOL, Bristol (GB)

(72) Inventors: Amy SMART, Bristol (GB); Olena DORAN, Bristol (GB); John Patrick HART, Bristol (GB); Adrian CREW, Bristol (GB)

(21) Appl. No.: 18/840,278

(22) PCT Filed: Feb. 20, 2023
(86) PCT No.: PCT/EP2023/054170

§ 371 Date: Aug. 21, 2024

(30) Foreign Application Priority Data
Feb. 21, 2022 (GB) 2202278.4

(51) Int. Cl. G01N 27/327; C12Q 1/00; C12Q 1/26; C12Q 1/61; G01N 27/30

(52) U.S. Cl. CPC G01N 27/3276; C12Q 1/002; C12Q 1/26; C12Q 1/61; G01N 27/308; G01N 27/3271; G01N 2333/90203; G01N 2333/92

(57) ABSTRACT

A biosensor for detecting triglycerides. The biosensor includes screen printed carbon electrodes (SPCEs), immobilised lipase and one or more other immobilised enzyme(s).

### FIELD OF INVENTION

The invention relates to the analysis of free fatty acids and triglycerides, particularly the detection and quantification of polyunsaturated, monounsaturated and saturated fatty acids in foodstuffs, bodily fluids and environmental samples.

### BACKGROUND TO THE INVENTION

Fatty acids, which predominantly occur as triglycerides, play an important role in food quality, health status and the environment. Measuring the fatty acid content of food, such as n-3 and n-6 polyunsaturated fatty acids (PUFAs) and saturated fatty acids, has implications for the quality and nutritional status of that sample, which is of interest to food producers and consumers. n-6 Linoleic acid (from trilinolein) is of particular interest, as it is one of two essential PUFAs which cannot be synthesised by the body and therefore must be ingested.

There is a need to develop effective technologies for the detection and quantification of triglycerides. These may be analysed by traditional chromatographic methods; however, these are expensive, time consuming and must be performed in a lab by skilled personnel.

Schoemaker 1997 describes a multi-step method to produce free linoleic acid from trilinolein, to be measured electrochemically (in a flow-cell). Schoemaker's multi-step method requires a skilled chemist with access to expensive laboratory reagents and equipment, and would take in excess of several hours. Furthermore, the concept of the biosensor does not feature in this paper, which uses a flow-cell as the final measurement step. Therefore, an obvious route to a biosensor would not be gleaned from this paper. Lipase and lipoxygenase are not used together. It is now over two decades since Shoemaker's paper has been published.

The patent literature describes the use of immobilised lipases for the analysis of triglycerides, for example WO2006/104077, CN107037102. In WO2006/104077 the fabrication method involves immobilisation of lipase and glycerol dehydrogenase onto the surface of an electrode. Similarly, in CN107037102 Immobilised lipase converts any and all triglycerides into free fatty acids and glycerol. There is no subsequent step, enzymatic or otherwise, that is capable of differentiating between the classes of triglyceride. Both prior art documents only measure total triglycerides (PUFAs, MUFAs (monounsaturated fatty acids) and SFAs (saturated fatty acids)), they cannot differentiate between these different classes of triglyceride. Being able to differentiate between different classes of triglycerides is important for example in the evaluation of food quality and microbial activity of soil.

The above demonstrates a pressing need for a rapid, simple, cost-effective and user-friendly technology for detection and quantification of fatty acids, for example in carcasses and meat at the point of test. In addition to the above, there are applications for this technology in the agri-food, biomedical and environmental sector, for example detecting microbial activity in soil, and in the medical and veterinary sector, detecting triglycerides in bodily fluids, such as blood.

### SUMMARY OF THE INVENTION

According to a first aspect, the present invention relates to a biosensor for detecting triglycerides, the biosensor comprising screen printed carbon electrodes (SPCEs), immobilised lipase and one or more other immobilised enzyme(s).

According to a second aspect, the present invention relates to an array of biosensors according to the first aspect, wherein the array preferably comprises three biosensors, one capable of detecting each of the three classes of triglycerides, MUFAs, PUFAs and SFAs.

According to a third aspect, the present invention relates to a method for detecting triglyceride comprising the steps of: a) contacting the biosensor of the first aspect with a sample comprising triglyceride; b) applying an amperometric or voltammetric waveform to the biosensor; and c) measuring the current response which is proportional to the concentration of fatty acid(s).

According to a fourth aspect, the present invention relates to the use of a biosensor according to the first aspect for the detection of triglycerides, preferably to measure the concentration of triglycerides in a sample.

The novel biosensor approach based on screen-printed carbon electrodes has many benefits; they can be manufactured in a wide-range of geometries at low cost as carbon is an inexpensive material, therefore they can be considered as disposable; these characteristics lead to rapid, portable and user-friendly devices. Electrocatalysts may be incorporated into the carbon ink of sensors where they act as electron shuttles for electrochemical reactions, thus increase the sensitivity and selectivity of the device. Further selectivity is achieved by incorporating a suitable enzyme onto the surface of the electrochemical transducer.

Immobilising the lipase leads to superior sensing compared to using lipase in free solution in terms of: (1) performance characteristics (limit of detection, linear range, co-efficient of variation). The inventors have shown good L.o.D. of 45.5 nM, linear range of 2 to 10 uM, and a better co-efficient of variation than using a LOX biosensor with lipase in free solution (5.05% vs 5.22%); (2) cost, since fewer enzyme units are used; and (3) time/simplicity, as there are fewer steps required in the measurement.

Previous work by the inventors has demonstrated the feasibility of using screen-printed carbon electrodes (SPCEs) in suitable formats as sensors and biosensors for the analysis of target analytes in challenging matrices; for example for agri-food applications including progesterone in milk, monosodium glutamate in stock cubes, fructose in fruit juice, organophosphates in cereals and raw produce, boar taint in pork, and thiamine in soft drinks. However, they have not been used before to detect or quantify triglycerides, particularly PUFAs.

The inventors have now developed a novel, screen-printed biosensor for the measurement of triglygerides, primarily through their conversion into linoleic acid. This has been successfully used to measure trilinolein in a food supplement, in a simple step, using multiple enzymes immobilised on the surface of the biosensor. Lipase in the biosensor is used to break down triglycerides into free fatty acids, which are measured using a selective enzyme (preferably LOX). For the measurement of polyunsaturated free fatty acids, LOX catalyses the oxidation of free PUFAs to the hydroperoxide form, which are measured with a screen-printed carbon electrode, preferably containing cobalt phthalocyanine.

It is preferred that one of the enzymes other than lipase in the biosensor is LOX. However, LOX is not essential. In biosensors that do not include LOX, the generated free fatty acid is measured by electrocatalytic oxidation at the CoPC-SPCE.

The present invention involves the novel immobilisation of lipase and an enzyme such as LOX onto a screen printed carbon electrode to create a biosensor for the measurement of triglyceride fatty acids. The sensor preferably contains a cobalt phthalocyanine (CoPC-SPCE).

In a preferred embodiment lipase generates glycerol and three free fatty acids from the triglyceride. LOX produces a free fatty acid hydroperoxide from the free fatty acids. This is electrocatalytically oxidised using cobalt phthalocyanine, which generates the response. This biosensor measures polyunsaturated fatty acids (PUFAs). Monounsaturated Fatty Acids (MUFAs) can be measured by adding a desaturase enzyme. Saturated fatty acids (SFAs) can be measured by adding two desaturase enzymes. Please see reaction scheme FIG. 1, below.

Other classes of triglycerides containing monounsaturated or saturated fatty acids may be selectively measured by incorporating additional selective enzymes onto the biosensor. Monounsaturated triglycerides (e.g. triolein) can be broken down into free fatty acids (e.g. oleic acid) using lipase, and then a delta-12 desaturase enzyme converts oleic acid into linoleic acid, where can be measured using LOX in the same way as detailed above. Saturated triglycerides (e.g. tristearin) containing stearic acid, can be broken down by lipase into stearic acid, and a delta-9 desaturase enzyme can break down stearic acid into oleic acid. The following steps are the same as detailed above. We have previously reported on an amperometric biosensor for the measurement of free linoleic acid itself (See Smart, A., Crew, A., Doran, O. and Hart, J. P., 2020. Studies Towards the Development of a Novel, Screen-Printed Carbon-Based, Biosensor for the Measurement of Polyunsaturated Fatty Acids. Applied Sciences, 10(21), p. 7779). This is for detecting free linoleic acid, not triglycerides.

### DETAILED DESCRIPTION

The present invention relates to a biosensor for detecting triglyceride fatty acids, preferably for measuring the concentration of triglyceride fatty acids in a sample. The biosensor comprises screen printed carbon electrodes (SPCEs), with lipase and one or more other enzymes immobilised separate from, onto or within the working electrode.

This is different to Schoemaker et al which involves the addition of lipase into the solution for analysis before the sensing step, and is limited to the measurement of trilinolein. The present invention creates an improved biosensor, which is able to measure two other classes of fatty acid triglycerides, 1) monounsaturated and 2) saturated fatty acid triglycerides. Furthermore, in the application of the present invention fewer steps are required for the measurement of fatty acid triglycerides, thereby creating efficiency gains in terms of time and cost.

By biosensor we mean a device which incorporates living organisms or biological molecules, in this case enzymes, to detect the presence of chemicals, in this case triglycerides/fatty acids.

The screen-printed carbon electrode usually contains two or preferably three electrodes. SPCEs are known to the person skilled in the art. The SPCEs preferably comprise an organometallic electrocatalyst in the carbon ink such as cobalt phthalocyanine, iron phthalocyanine, nickel phthalocyanine or copper phthalocyanine. Usually the SPCEs are cobalt phthalocyanine SPCEs (CoPC-SPCEs).

The triglycerides that the biosensor can detect, and quantifiably measure, generally include trilinolein, triolein and tristearin or a combination thereof. Usually the free fatty acids that are generated from the triglycerides include linoleic acid, alpha-linolenic acid, oleic acid, stearic acid, or a combination thereof, but can also include fatty acids such as arachidonic acid and palmitic acid etc.

For the measurement of polyunsaturated fatty acids (PUFAs), lipase and lipoxygenase enzymes are included. When the triglyceride that is detected or measured is trilinolein, the immobilised enzymes are lipase and lipoxygenase.

For the measurement of monounsaturated fatty acids (MUFAs), lipase, lipoxygenase and a desaturase enzyme is included.

When the triglyceride that is measured is triolein, the immobilised enzymes are lipase, Delta-12 desaturase and lipoxygenase.

For the measurement of saturated fatty acids (SFAs), lipase, lipoxygenase and two desaturase enzymes are included. When the triglyceride that is detected is tristearin, the immobilised enzymes are lipase, Delta-12 desaturase, Delta-9 desaturase and lipoxygenase.

A key benefit of this approach is that the biosensor can discriminate these three classes of triglyceride, which is not possible with other types of triglyceride biosensors.

The biosensors comprise SPCEs, the immobilised lipase and other immobilised enzyme(s). The immobilised lipase and other enzymes(s) are usually in one or more layers which are separate from, on, or within the SPCE. Preferably the lipase and other enzyme(s) are immobilised in the same layer. This is usually the layer that is closest to, on or within the SPCE.

There are three possible methods of enzyme immobilisation:
1. Drop-coating onto the working electrode;
2. Drop-coating onto a perm selective membrane, such as cellulose acetate, which covers the working electrode; or
3. Including enzymes in a carbon ink formulation and screen-printing this mixture onto a substrate, such as plastic.

Wherein drop-coating, a cross-linking agent, which is preferably glutaraldehyde, may be included as the outermost layer.

For the drop-coating method, enzymes may be deposited in one or more layers, added sequentially.

Multiple biosensors may be combined together in an array. There are preferably 2 or 3 to 30 biosensors in an array, and can be 3 to 6. The biosensors in the array can all be the same type but are preferably different types. Preferably the array consists of three biosensors, one for each of the three classes of triglycerides, MUFAs, PUFAs and SFAs. The biosensors in an array are usually arranged with a back-back configuration or a comb configuration.

In the method for detecting or measuring triglyceride, the first step a) is to contact the biosensor with a sample comprising triglyceride. The triglyceride can be in a solid sample, a semi-solid sample or a liquid sample. Any sample can be tested using the biosensor, but it is particularly valuable to test a sample that is from or is an animal carcass, is human or animal bodily fluid such as blood, or is soil.

The second step, b), is to apply an amperometric or voltammetric waveform to the biosensor, such as square wave voltammetry or differential pulse voltammetry. The values are dependent on the sample type, but when the technique used is amperometry in stirred solution, the operating potential of the biosensor is usually from about +0.4 V to about +0.8V vs. Ag/AgCl, preferably about +0.5 V vs. Ag/AgCl.

The third step, c), is the measuring the current response. This will be proportional to the concentration of fatty acid(s). Accordingly, this allows the concentration of triglycerides in the sample to be determined.

This method/use of the biosensor has many applications. Where the sample is food, it can detect and monitor the quality of the food. Where the sample is blood or another bodily fluid, it can be used to determine and monitor health. Where the sample is soil, it can be used to monitor or detect the quality of the soil, including faecal contamination or microbial activity.

The sample can be such that the concentration of triglyceride in the sample is 0.01 to 100 mM, or 0.5 to 50 mM and is preferably 0.2 to 10 mM.

### DESCRIPTION OF THE FIGURES

FIG. 1. Reaction scheme of lipase-lox biosensor according to the invention. This shows the SPCE, a Lipase and LOX layer closest to the SPCE, and a GLA layer outermost from the SPCE. In this the triglyceride (R) passes through the GLA layer and reacts with the LOX-Lipase layer. The lipase generates glycerol and a free fatty acid (R(FFA)) from the triglyceride. LOX produces a free fatty acid hydroperoxide (ROOH) from the free fatty acid. This is electrocatalytically oxidised using cobalt phthalocyanine, which generates the response.

FIG. 2. Biosensor array according to the invention. Scheme showing the conversion of different triglycerides into linoleic acid which occurs during the operation of the biosensor array. Square blocks are enzymes which are immobilised in the biosensor. In the case of polyunsaturated trilinolein (A) this is converted into linoleic acid by lipase, which is oxidised by lipoxygenase to linoleic hydroperoxide. This species undergoes electrocatalytic oxidation, at the underlying CoPC-SPCE, to produce the analytical response. For the monounsaturated triglyceride triolein (B), this is converted to oleic acid by lipase. Oleic acid is converted to linoleic acid by the desaturase enzyme Delta-12. This undergoes the reaction described previously to produce the analytical response. In the case of tristearin (C), this is enzymatically converted by delta-9 desaturase to oleic acid. This undergoes the same reactions as mentioned previously for oleic acid to produce the analytical response.

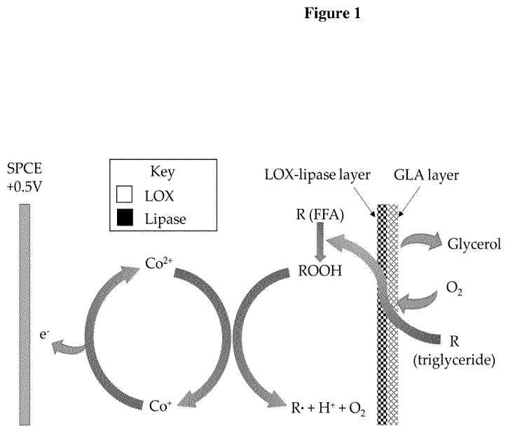

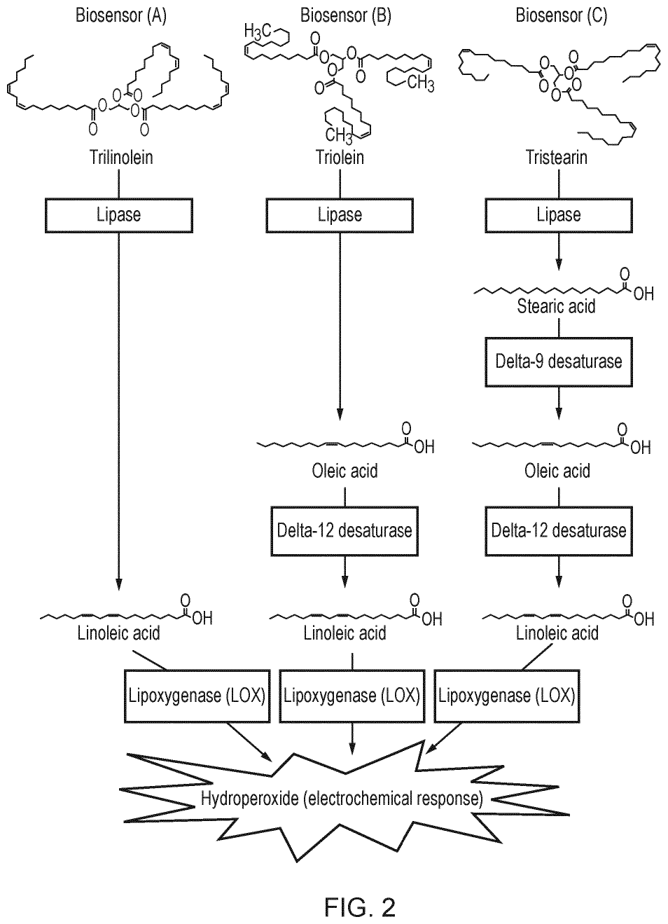

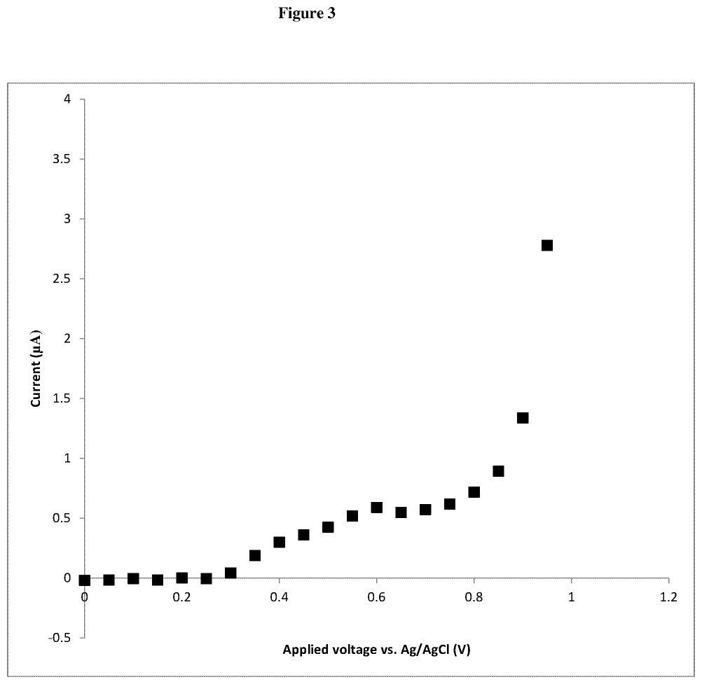

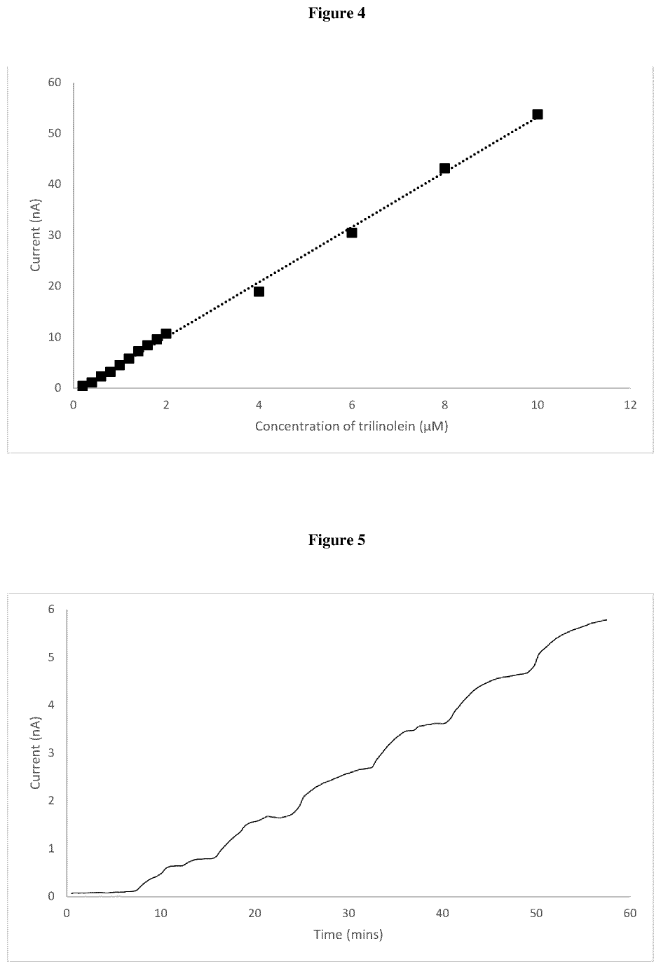

The reaction schemes in FIGS. 1 and 2 are novel during the operation of a biosensor for triglyceride measurement. The reactions in FIG. 1 describes in more detail the reaction sequence shown in FIG. 2 (biosensor A). FIG. 2 (biosensor B) includes an additional enzyme (delta-12 desaturase, which inserts a double bond between carbon number 12 and 13, counting from the carboxyl end of the molecule). FIG. 2 (biosensor C) includes an additional desaturase (delta-9 desaturase, which inserts a double bond between carbon number 9 and 10, counting from the carboxyl end of the molecule). The reaction schemes in FIG. 2 are crucial to the invention as it allows discrimination between the classes of fatty acid triglyceride when measured simultaneously. These are original schemes in the context of biosensors.

FIG. 3. Hydrodynamic voltammogram of a CoPC-SPCE biosensor containing 15 units of LOX and 45 units of lipase, in a 33.3 μM solution of trilinolein.

FIG. 4. Calibration plot of trilinolein using a CoPC-SPCE biosensor with 45 units of lipase and 45 units of LOX, in conjunction with amperometry in stirred solution at +0.5 V vs. Ag/AgCl

FIG. 5. Amperogram showing individual additions of trilinolein, using a CoPC-SPCE biosensor with 45 units of lipase and 45 units of LOX, in conjunction with amperometry in stirred solution at +0.5 V vs. Ag/AgCl

### EXAMPLES

### 1.1. Instrumentation

All voltammetric and amperometric measurements were carried out with a μAutolab III potentiostat interfaced to a PC for data acquisition via NOVA v2.0 (Metrohm, Barendrecht, The Netherlands) or an AMEL Model 466 polarographic analyser attached to an ABB Gorez SE120 chart recorder. An in-house low pass filter (time constant 22 s) was incorporated between the potentiostat and the chart recorder to substantially reduce stirrer noise.

CoPC-SPCEs are commercially available and were supplied by Gwent Electronic Materials Ltd. (Pontypool, UK). The working electrode was fabricated using a carbon-based ink with CoPC (C2030408P3) and the reference electrode was fabricated using a Ag/AgCl ink (C2130809D5). The working electrode area (3 mm×3 mm) was defined using electrical insulation tape.

All pH measurements were performed using a Testo 205 (Testo Limited, Alton, Hampshire UK) pH meter. Solutions were stirred using a colour squid (IKA, Tunbridge Wells, UK) and warmed using a HAAKE P5 water bath (Thermo Scientific, Loughborough, UK).

Surface morphology and composition of the working electrode were analysed using a Quanta FEG 650 scanning electron microscope (FEI, Hillsboro, OR, USA) (4000× magnification; samples were gold-coated).

### 1.2. Chemicals and Reagents

Conjugated linoleic acid (CLA) capsules were purchased from Holland and Barrett; five capsules were opened and their contents mixed. All other chemicals were purchased from Sigma Aldrich (Dorset, UK). Deionised water was obtained from a Purite RO200 Stillplus HP System (Oxon, UK). Stock solutions of monosodium, disodium and trisodium orthophosphate were prepared at a concentration of 0.2 M by dissolving the appropriate mass in deionised water; these were then titrated to achieve the desired pH and diluted in the cell to achieve a working concentration of 0.1 M. Sodium chloride was prepared to a concentration of 1.0 M by dissolving the appropriate mass in deionised water; this was diluted in the cell, giving a final concentration of 0.1 M.

Aliquots of LOX and lipase solutions were diluted with 0.1 M pH7 phosphate buffer saline to give the desired number of enzyme units. A 50% glutaraldehyde stock solution was diluted with 0.1M pH7 phosphate buffer saline give a 0.01% solution.

Stock solutions of trilinolein and CLA from capsules were prepared by dissolving the required mass in ethanol to achieve 1 mM solutions.

A 1 mM linoleic acid stock was prepared by dissolving the desired mass in methanol.

### 1.3. Biosensor Fabrication and Storage

To make LOX-lipase biosensors, CoPC-SPCE working electrodes were drop-coated with 10 μl of enzyme solution, containing a) 15 U of LOX, b) 45 U of lipase, or c) 15 U of LOX and 45 U of lipase mixed together. Each enzyme layer was dried overnight using a desiccator under vacuum. Electrodes a) and b) were further drop-coated with 10 μl of enzyme solution containing 45 U of lipase or 15 U of LOX, respectively to make a second layer. All electrodes contained 15 U of LOX and 45 U of lipase.

Enzyme was cross-linked to the electrode surface by drop-coating 10 μl of 0.01% glutaraldehyde solution, which was also dried overnight using a desiccator under vacuum. Biosensors were stored in airtight containers at 4° C. for up to 4 months.

### 1.4. Amperometric and Voltammetric Procedures

In order to deduce the optimum operating potential for amperometric measurements in stirred solution using the LOX-lipase biosensor, a hydrodynamic voltammogram was constructed over the range +0.0 to +1.2 V vs. Ag/AgCl using 100 UM of linoleic acid (from 33.3 μM of trilinolein) in 10 ml 0.1 M pH8 phosphate buffer saline. The solution was warmed to 37° C. and stirred at 250 rpm.

A calibration study was performed with the LOX-lipase biosensor in conjunction with amperometry in stirred solution at +0.5 V vs. Ag/AgCl. Ten 20 μL additions of 1 mM trilinolein were made into a cell containing 10 mL pH 8 0.1 M phosphate buffer saline, stirred at 250 rpm at 37° C. A low concentration calibration study was performed using an analogue instrument with a low pass filter to reduce stirrer noise. Ten 2 μL additions of 1 mM trilinolein were added into a cell containing 10 mL 0.1 M pH 8 phosphate buffer saline solution, stirred at 250 rpm and warmed to 37° C.

Standard addition was used to calculate the percentage recovery of trilinolein from CLA capsules that could be achieved using the LOX-lipase biosensor. A cell was prepared with 10 ml 0.1M pH8 phosphate buffer saline. Amperometry in stirred solution was performed at +0.5 V vs. Ag/AgCl, and the cell was stirred at 250 rpm and warmed to 37° C. A 1 mM trilinolein solution from CLA capsules was pipetted into the cell, followed by five additions of 1 mM linoleic acid.

The effect of storage on LOX-lipase biosensor performance was assessed by performing calibration studies using the biosensors stored for different lengths of time. Five 20 μl additions of 1 mM trilinolein were made into 10 ml 0.1 M pH8 phosphate buffer saline; amperometry in stirred solution was used in conjunction with a CoPC-SPCE containing 45 U of lipase and 15 U of LOX, at 0.5V vs. Ag/AgCl, 37° C. and 250 rpm.

### 1.5. Fabrication and Evaluation of a LOX-Lipase Biosensor

Lipase (in excess) was combined with LOX onto a base CoPC-SPCE transducer in three different fabrication methods: 1) lipase layer, then LOX layer, then glutaraldehyde layer; 2) LOX layer then lipase layer then glutaraldehyde layer, and 3) a mixed LOX-lipase layer then glutaraldehyde layer. All three biosensors contained 15 units of LOX and 45 units of lipase.

The three different biosensors were evaluated by carrying out calibration studies over the range 2 to 10 μM of trilinolein, using amperometry in stirred solution at +0.5 V vs Ag/AgCl. Further biosensors were prepared by the method involving the deposition of the mixture comprising LOX and lipase onto the CoPC-SPCE. A linear relationship was observed between concentration of trilinolein and current response, demonstrating that the biosensor can be used to directly measure trilinolein in solution, avoiding the need to add lipase to the solution. The proposed reaction scheme is shown in FIG. 1.

A biosensor array has been designed to simultaneously measure three classes of triglycerides (saturated, monounsaturated and polyunsaturated), based on the biosensor described above. FIG. 2 shows the sequence of enzyme reactions that occur during the operation of the electrochemical biosensor for three different fatty acid triglycerides as described above.

In order to measure the three individual classes of fatty acid triglycerides in a mixture, an array consisting of the three biosensors shown in FIG. 2 may be simultaneously applied to a single sample. This would produce three electrochemical responses which can then be used to deduce the correct response for each fatty acid: biosensor (A) measures trilinolein only; biosensor (B) measures both trilinolein and triolein (triolein can be measured by deducting the response obtained at (A) from response (B); and biosensor (C) measures tristearin, triolein and trilinolein (tristearin can be measured by deducing response (B) from response (C)).

Scanning electron microscopy was used to investigate the surface morphology of the selected LOX-lipase biosensor, and a cohesive outer film can be seen to be present, which is attributed to the cross-linking agent glutaraldehyde. The porous nature of the glutaraldehyde allows ingress of the analyte but retains the enzymes within the reaction layer; this is indictaed by the steady state responses, see FIG. 5.

Hydrodynamic voltammetry was performed with the LOX-lipase CoPC-SPCE biosensor. The hydrodynamic voltammogram was performed using the same final concentration of linoleic acid as before (33.3 μM of trilinolein producing 100 μM of free linoleic acid). A broad plateau from about +0.4 V to +0.8 V vs. Ag/AgCl was observed (FIG. 3). Consequently the operating potential of +0.5 V vs. Ag/AgCl was selected for further work.

### 1.6 Performance Characteristics of the LOX-Lipase Biosensor

To investigate the possibility of extending the linear range of the LOX-lipase biosensor with trilinolein, an analogue instrument paired with a low-pass filter was used to eliminate stirrer noise; this instrumental setup was used to perform a low calibration study over the range 0.2 to 10 mM trilinolein. Amperometry in stirred solution was performed at +0.5 V vs Ag/AgCl. The resulting extended calibration plot is shown in FIG. 4. The plot shows a linear relationship between concentration of trilinolein and amperometric response over a concentration range of 0.2 to 10 M of trilinolein. The limit of detection can also be deduced from measuring 3 times the noise of the raw amperometric data (FIG. 5), and this was calculated at 45.5 nM. These performance characteristics compare very favourably with other lipase-containing biosensors which have previously been developed for triglyceride determination.

The LOX-lipase biosensor was investigated for the determination of trilinolein in a more complex matrix, in CLA capsules. A standard addition method was used in conjunction with amperometry in stirred solution at +0.5 V vs Ag/AgCl (Table 1). The percentage recovery was very good, averaging 86%. The co-efficient of variation was also low at 5.05%, i.e. very good reproducibility.

TABLE 1
Percentage recovery of trilinolein in capsules using (bio)sensor

| | THEORETICAL | MEASURED | % |
|---|---|---|---|
| N | CONC. | CONC. | RECOVERY |
| 1 | 3.0 μM* | 2.6 μM | 86.7 |
| 2 | 3.0 μM* | 2.5 μM | 83.3 |
| 3 | 3.0 μM* | 2.5 μM | 83.3 |
| 4 | 3.0 μM* | 2.8 μM | 93.3 |
| 5 | 3.0 μM* | 2.5 μM | 83.3 |
| MEAN | | | 86.0 |
| SD | | | 4.35 |
| COV | | | 5.05 |

*Theoretical concentration of trilinolein from contents of capsules which manufacturers claim is 80% conjugated linoleic acid

### 1.7 Storage Study

The performance of the biosensor over time was assessed by performing calibration studies of linoleic acid at monthly intervals over a period of 4 months of storage in a refrigerator (4° C.) following fabrication. After 4 months of storage, there was no decrease in sensitivity; the slopes at each time point were not statistically significantly different from each other using a two-tailed t-test (p value was greater than 0.05). The linear range was also the same over each time point (2 to 10 μM).

### CONCLUSION

A novel biosensor of the invention was successfully used to measure linoleic acid, obtained from hydrolysed trilinolein (using lipase) which was present in a commercially available pharmaceutical supplement. The triglyceride biosensor was fabricated by immobilising lipase with LOX into the reaction layer, on the surface of a CoPC-SPCE. The novel biosensor showed favourable performance characteristics for trilinolein measurement, with a wide linear range of 0.2 to 2 μM, and a low limit of detection of 45.5 nM. The novel biosensor was successfully applied to the determination of trilinolein in a pharmaceutical food supplement; the average recovery was 86.0% with a corresponding coefficient of variation of 5.05%. The new trilinolein biosensor may be applied to a range of other food types, and has potential for clinical analysis. The storage stability data and high reproducibility make these devices attractive for commercialisation. This LOX-lipase biosensor, which measures trilinolein, can be used as part of a biosensor array which is able to selectively measure poly-, mono- and saturated triglycerides. This can be achieved by including additional biosensors, fabricated by incorporating suitable desaturase enzymes onto the LOX-lipase biosensor.

1. A biosensor for detecting triglycerides, the biosensor comprising screen printed carbon electrodes (SPCEs), immobilised lipase and one or more other immobilised enzyme(s).

2. The biosensor of claim 1, wherein the immobilised lipase and the one or more other immobilised enzyme(s) are immobilised in one or more layers which are separate from, on, or within the SPCE.

3. The biosensor of claim 1, wherein the triglycerides that the biosensor can detect are selected from trilinolein, triolein, tristearin or a combination thereof.

4. The biosensor of claim 1, wherein the triglycerides that the biosensor can detect comprise linoleic acid, alpha-linolenic acid, oleic acid, stearic acid, or a combination thereof.

5. The biosensor of claim 1, wherein the one or more other immobilised enzyme(s) are selected from lipoxygenase (LOX), delta-12 desaturase, Delta-9 desaturase, or a combination thereof.

6. The biosensor of claim 1, wherein the triglyceride that is detected is trilinolein, and the one or more immobilised enzyme is LOX.

7. The biosensor of claim 1, wherein the triglyceride that is detected is triolein and the one or more immobilised enzymes are LOX and delta-12 desaturase.

8. The biosensor of claim 1, wherein the triglyceride that is detected is tristearin and the one or more immobilised enzymes are LOX, delta-12 desaturase and Delta-9 desaturase.

9. The biosensor of claim 1, further comprising a cross-linking agent which is glutaraldehyde.

10. The biosensor of claim 1, further comprising an electrocatalyst selected from cobalt phthalocyanine iron phthalocyanine, nickel phthalocyanine or copper phthalocyanine.

11. An array of biosensors according to claim 1, wherein the array comprises three biosensors, one capable of detecting each of the three classes of triglycerides, MUFAs, PUFAs and SFAs.

12. An array of biosensors according to claim 11, wherein the biosensors are arranged in the array with a back-back configuration or a comb configuration.

13. A method for detecting triglyceride comprising the steps of:
a) contacting the biosensor of claim 1 with a sample comprising triglyceride;
b) applying an amperometric or voltammetric waveform to the biosensor; and
c) measuring the current response which is proportional to the concentration of fatty acid(s).

14. The method of claim 13, wherein the triglyceride is in a solid sample, a semi-solid sample or a liquid sample.

15. The method of claim 13, wherein the operating potential of the biosensor is from about +0.4 V to about +0.8V vs. Ag/AgCl.

16. (canceled)

17. (canceled)
